(19) United States
(12) Patent Application Publication
(10) Pub. No.: US 2016/0158541 A1
(43) Pub. Date: Jun. 9, 2016

(54) METHOD AND DEVICE FOR GENERATING SFERICS ALTERNATING FIELD EMISSIONS HAVING MINIMAL METEOROTROPIC OR BIOTROPIC EFFECT ON HUMANS ETC.

(71) Applicant: FLORIAN KÖNIG ENTERPRISES GMBH, Germering (DE)

(72) Inventor: FLORIAN M. KÖNIG, GERMERING (DE)

(21) Appl. No.: 14/903,984

(22) PCT Filed: Jul. 11, 2014
(86) PCT No.: PCT/DE2014/000352

§ 371 Date: Jan. 8, 2016

(30) Foreign Application Priority Data
Jul. 11, 2013 (DE) 10 2013 011 560.4

(51) Int. Cl. A61N 1/16

(52) U.S. Cl. CPC A61N 1/16

(57) ABSTRACT

The invention relates to a method and to a device for generating sferics alternating field emissions having a minimal biotropic effect for optimised application of alternating-field-type radiation to humans, animals and plants compared to naturally occurring environmental sferics that are weather-dependent or attenuated outdoors or indoors, e.g. biotropic, and also technics or radiation forms originating from human technology, which are unfortunately largely too intensive, in particular in current times; firstly, in particular one or more alternating signals attributable to or based on natural, atmospheric impulse discharge processes in the signal propagation forms thereof are post-processed with regard to interfering, periodic duration/signal components of technical origin that are to be deleted and in addition individual impulses that are too intensive in signal-dynamic, level-related terms are deleted; secondly, said post-processed alternating signals are mixed, preferably with or without other, selected, aperiodic/periodic/impulse-like alternating signal forms are mixed; and thirdly and finally, the preferably mixed, ideal total alternating signal is radiated or emitted via alternating field transmission means such that said signal is adapted in a level-controlled manner.

### CROSS-REFERENCE TO RELATED APPLICATIONS

The present application claims priority to PCT International Application No. PCT/DE2014/000352 filed on Jul. 12, 2014, which application claims priority to German Patent Application No. 10 2013 011 560.4 filed Jul. 11, 2013, the entirety of the disclosures of which are expressly incorporated herein by reference.

### STATEMENT RE: FEDERALLY SPONSORED RESEARCH/DEVELOPMENT

Not Applicable

### FIELD OF THE INVENTION

The invention relates to a method and to a device for generating sferic alternating field emissions having a minimal meteorotropic or biotropic effect (BMSE) on humans or biological organisms (including plants).

### DESCRIPTION OF THE PRIOR ART

It is sufficiently known that a pulsed alternating field has accompanied the earth's atmosphere and weather activity since its beginning. The field appears to have played a crucial role in the evolution of life on earth and previously was dismissed as background noise. Also dismissed as esoterics were those who observed, analyzed and came to conclusions about this field as atmospherics or sferics. On the contrary, it has escaped the notice of meteorologists and electrophysicists that, in addition to the continuous oscillations known as Schumann resonances (7.83 Hz with harmonic spectrum; cf. “Unsichtbare Umwelt” [“Invisible Environment”], Book 1986+2012, Herbert L. König) on our planet, the original generators of this oscillation—namely lightning or dark field discharges (see Friese sferics systems and literature) etc.—have essentially gone unnoticed in the scientific literature. In the meantime, biostatistical results of meteoropathy tests have confirmed that these sferics penetrate undetected into enclosed areas of our main living rooms in Central Europe and are the cause of biotropic effects such as migraines, head/scar pains, concentration disorders, digestion problems, tinnitus, aggressive behavior or fatigue to the point of depression etc. It is not to be forgotten that, in addition to such a biotropic effect, the unrecognized connection between such sferics and the critical envelope shapes on neuronal couplings (as can be found in magnetic field therapy and secondary sferics such as reference radiation) are to be considered; this must especially not be forgotten by technical generators of field emitters (technics senders), like those arbitrarily realized by engineers without considering [the effect of] their radiation characteristics on the increasing number of electrosensitive (and weather-sensitive) individuals. Once again it arises: how can it be that weather-sensitivity exists inside residential buildings and offices when tests of meteorotropic variations in temperature, air pressure, humidity, etc., are supposed to prove the non-existence of weather-sensitivity, which, however, exists especially in air-conditioned spaces as well as “outside”! Consequently, this must be an information transfer medium that does not fit into meteorological parameters, i.e. sferics. A modeling of the transfer function of individual sferic pulses to the recipient can have consequences for communications technology such that each weather or storm impulse up to the location of the recipient (see people as receiving antennae) conceals within it the sum of all weather events passing through the atmosphere; like a kind of fingerprint, even if this impulse for the most part is significantly weaker than, for example, near-field thunderstorm discharges when it arrives at the recipient! It can be precisely concluded from from the most recent scientific findings that, within a few minutes of impact time, a masking of nowcast sferics via sferic signal recordings recorded at a suitable location and signaling technology sferic signal recordings revised according to the invention without technics (thus those with ultimately stronger level reproduction at a human receiver) modify neuronal and meridian-related feedback measurement values of all organ values towards a state of wellbeing, relaxation, general health and away from all contrary values caused by multiple stresses (see weather and technics stress).

After many years of research on humans, it is now possible to produce a kind of extract of the knowledge about the use of sferics as low-frequency reference radiation, ideal modulation envelope curve emitters (see, among others, biocompatible technics of new types of electrical loads) or as a therapeutic agent that generates minimal meteorotropic and biotropic effects; in other words, there is no global, ideal weather condition unaffected by technics that simultaneously and unambiguously has a positive effect on all the different kinds of people in all areas of the world (see reference radiation)! The application by Hengstenberg (DE4414235A1) as well as those that are ascribed to the inventor always start from the ideal or, respectively, the assumption of technical applicability of additive sferic field emissions obtained directly from the Earth's atmosphere and generated by means of technical transmission/emission devices, which gives us nature as the evolutionary reference radiation.

However, regarding continuative, more recent research work and results (see also the aforementioned inventors of earlier sferic field emission devices and their commendable basic research), in the optimized application or technical reproduction of this 1:1 alternating field reproduction from nature, a minor but significant error has unfortunately occurred that has only been caught by the most modern, high-resolution statistical sferic signal analysis methods and related tests on test subjects within the framework of effect optimization, and with the present invention it is proposed as a preferred applicable method: small, infrequent “micro-bolts that contaminate the ideal sferic therapy signal,” a non-matching signal strength paired with a “false sferic signal dynamic”, and interfering signals of a technical nature (for roughly the last 80 years, i.e. before the development of DE4414235A1) reduce a (statistically) positive relaxation/health effect of additive emitted sferic alternating fields across the largest possible group of test subjects. In other words, from (blind test scheme) tests of test subjects from 2002 to 2012 it turns out that original sferic emissions were conditionally effective, perhaps even ineffective, when the level comparison ratio (cf. weather online sferics with additive generated sferic fields) and its impulse-technical signal dynamic behavior were not controlled inside certain limits; moreover, according to the influence of technics, they were not emitted without interference (see Hengstenberg & applicant). It should not be forgotten that, from the inventor's tests on groups of test subjects, an additively mixed signal—preferably a signal stochastically distributed according to a chronological envelope—outside of the sferics-typical frequency range was offered to the test subjects, resulting in faster and more profound effects. Audio-signal engineering methods (see dynamic compression) and inferior data-compromising mp3 signal conservation (cf. recording/reproduction) must be avoided as insufficient and signal-distorting; see reduction of the positive effect on humans and thus generation of “technics in turn,” which the reference radiation conditions on the planet Earth—due to the slow processes of evolution (over millions of years)—are utterly (necessarily) counterproductive to daily life! Only a targeted processing of such sferic signals will make possible the most positive quality of the effect of sferics based on a signal-masking medium for negating the effect of commonplace nowcast/online sferics and technics on humans, animals or plants.

We now intend to briefly discuss here the issues and specifics of electrosmog impact factors on humans: our environment is burdened by numerous alternating electromagnetic fields. A large portion of these alternating electromagnetic fields is artificial, i.e. of human origin. One current prominent example of such fields are artificially created electromagnetic fields for transmitting data up to the gigahertz range. One example of this is the mobile telephone, what is known as the cell phone, which works in the GSM data transmission format and which in the future will also work in the UMTS data transmission format. These electromagnetic fields typically exhibit pulsing in the range from 100 Hz or, respectively, 217 Hz in the case of cell phones. Associated with this is severe electro-stress, which negatively affects organic cells. In the case of cell phones in particular, significant biological stress on organic cells of the user is unavoidable due to the high transmission power of these devices when the respective handheld device is used near the ear of the user. Liebrecht von Klitzing has stated in scientific publications and, among other places, in the magazine 2000 plus, No. 144 (12-1999, page 74), that the high dynamics of more than 60 dB is a trigger for severe cell irritations between the pulse packets of RF signal transmission, at a typical cell resonance of up to 400 Hz. This article also states that signal periodicity is an important factor in the impact on cells, and that a stochastic signal behavior is much better for organic cells because it is less damaging. There is also sufficient knowledge available about unfavorable biological effects of artificial high-frequency fields on organic cells of organisms, especially humans (see, for example, Herbert L. König, “unsichtbare Umwelt” [“invisible environment”], Munich, 1986).

However, stress on the human body also exists in the form of natural alternating electromagnetic fields. For example, it is known that certain weather situations, for example dry conditions in the Alps, are associated with naturally occurring alternating electromagnetic fields, which propagate horizontally and/or vertically and range between a few hundred Hz up to typically 50,000 Hz. Such “bad-weather fields” negatively affect organisms in a known manner (see “hairdryer,” for example). In contrast to this, it is known from “fair-weather fields” (also called “sferics”) that they have a positive effect on organisms. These naturally-occurring alternating electromagnetic fields range from a few hundred Hz up to typically at least 20 kHz and—in contrast to bad-weather fields—demonstrate a different amplitude and frequency distribution.

For the stimulation of the human organism and to compensate for certain unfavorable effects on it, as well as for calming and in general to compensate for electrosmog, it is known to use a battery-driven handheld device that generates artificial alternating electromagnetic fields in the lowest frequency range between 1.4 and 32.4 Hz (“MEDISENT,” www.magnet-feldtherapie-2000.de). This device does not take into account higher-frequency alternating electromagnetic fields, such as, for example, electrosmog in the audiofrequency range above 30 Hz and in the HF range.

### SUMMARY OF THE INVENTION

Therefore, one object of the present invention is to develop a device by means of which the electro-stress impacting a user can, at a minimum, be compensated for at least essentially independently of amplitude and frequency and/or by means of which the health of a user may be positively influenced.

This object is achieved by the features of claim 1. Advantageous developments of the invention are described in the dependent claims.

Accordingly, the present invention has developed a method and device for generating sferic alternating field emissions having a minimal meteorotropic or biotropic effect on (among other things) humans (also animals and plants), not to mention reducing damaging, neuronally very sensitive types of radiation of human origin that have negative effects, including among others today's wireless communications technologies with their modulators (in addition to the actual so-called “carrier”) and network/power supply/utilization devices of all kinds (see path of their current, current form up to electrical loads with harmonic wave alternating field spectra in the biological living environment of the Earth) in the frequency range under 100,000 Hz. It should be emphasized from this that the use of an original, pure “fair-weather field”, or one that nowadays can be taken directly from the Earth's atmosphere or, respectively, which—according to the invention—can first be basically recorded, often (also earlier by the inventor; see below immediately following according to the cited patent files and concluding remarks) preferably for the use of a prescribed, pure “fair-weather field” (or its sferic impulse signal sequences of natural atmospheric origin) should not be used directly or “ideally” for humans (or animals and plants) because IN THE FIRST PLACE there are, since the start of the 20th century, no longer any sferics that are “clean” or “unaffected by urban electrotechnology” which may be used harmlessly and be separately (synthetically/artificially additively) emitted at any location at any time. HOWEVER, SECONDLY and significantly more exactly here, the present invention—in contrast to earlier inventions, for example DE 4414235A1 or the patent family around EP 1354611A3 and U.S. Pat. No. 6,888,486B2—represents a novel approach because:

Recent epidemiologically germane tests (e.g., sferics impact tests within the scope of a weather-sensitivity study per ISBN No. 978-3-00-045904-7) and developments of the method according to the invention, as well as device applications (not only according to claim 1) give indications of how a nearly natural fair-weather field noise signal mixture—for example, as described herein—could, in application on test subject groups, have significant positive as well as stronger (see stimulating) neuronal or physical effects, in terms of the quality of its effect, on the body of a living organism (see test subjects) at rest. In the book “Die Natur Braucht Chaos” [“Nature Needs Chaos”] (author=inventor; see ISBN No. 978-3-89539-712-7), this could not yet be conclusively established or shown in the tests described using ONLY additive synthetic sferic alternating field emissions on test subjects. Thus in conclusion, the procedures according to the invention are to detect or, respectively, define impulse signal sequences captured or, respectively, registered from nature during suitable weather conditions as well as subsequent signal analyses of each too-strong (in terms of its level), statistically rarer single pulse (with malignant effect on human/animal health), and thereafter to excise or, respectively, eliminate these from the sferic time-signal sequences. The now “ideal” generated, pulsed alternating signal/recording corresponds to an “ideal high-pressure area” and its offered fair-weather field without individual disruption impulses (and preferably without technics).

In conclusion, according to the invention this “ideal” alternating signal/recording is mixed with one and/or more such “ideal fair-weather fields” and/or one or more aperiodic alternating signals (as in typological noises, for instance) and/or one or more periodic alternating signals, like those that in past decades were medically shown or recommended as therapeutically beneficial for selected applications. The reason for such a mixing of signals is the permanent, modern broadband presence of technics, which thus are only covered at the level of signal technology and thus in their (medically detectable) effect on organisms; also contemplated is the sharply increasing number of electrosensitive individuals which this should also help; according to a representative study by Peter Hoppe (from 2002; entitled “Prävalenz der Wetterfühligkeit” [“Prevalence of weather-sensitivity”], approx. 54% of people in Germany are weather-sensitive.

An additional note for general understanding: sferics represent not only a reference radiation element in the electromagnetic low-frequency range of our planet Earth (including the atmosphere) that has existed for millions of years, but rather appear to be a co- or sub-factor for triggering weather sensitivity unwellness. Last but not least, biologists recognize that animals (among others) retreat or seek protection from approaching storms or rainy areas of low pressure (cf. behavioral research on deer or ants). This doesn't happen during sunny fair weather; thus, no warning signals/alternating fields exist in the earth's atmosphere! Unfortunately, to date sferics have been misunderstood scientifically, for example, as so-called “meaningless background noise.”

According to an especially advantageous development of the invention already comprises a means to emit the signal made available according to the invention, prepared first by signal technology, and thereafter the consequently created alternating electromagnetic fields in the environment of a user and/or exposed biological organism, via available transmitting antenna means.

### BRIEF DESCRIPTION OF THE DRAWINGS

The invention is explained in detail below by way of example, using the drawings.

FIG. 1 is a block diagram of the method according to the invention and related devices;

FIG. 2 is an image of two linear (chronologically) alternating signal curves A1/A2, depending on the time t of various recorded sferic signals for a signal detail comparison, which signal-time amplitude curve ranges are usable or unusable and belong to revised according to the method and procedures according to FIG. 1, in order to be able to ultimately construct and emit an “ideal fair-weather field noise mixture” masking an ambient alternating field (see sferics & technics).

### DETAILED DESCRIPTION OF THE PREFERRED EMBODIMENTS

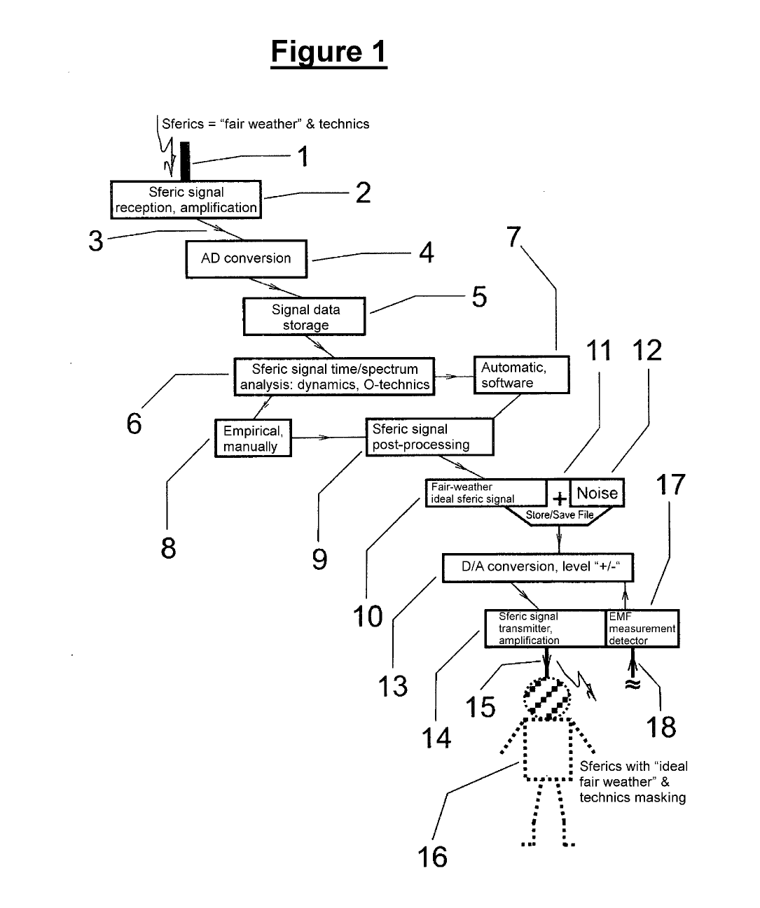

As is clear from the block diagram of FIG. 1, the method according to the invention includes all device elements for the generation close to the body of a natural alternating electromagnetic field, including
an receiving antenna 1 (coupling of low-frequency, impulse-heavy sferic alternating fields during a selected high-pressure area)
with a subsequent (see reference number or element 3, “subsequent step/direction indicator in the method), broadband, adjustable signal amplification device 2 for suitable predominantly present “fair-weather sferics” prevailing in the selected environment and which are to be recorded or registered, followed serially by an analog-to-digital converter 4 for the generation of data to be processed later from the sferic fair-weather field signal at rest;
followed in the method by a necessary storage 5 of data or data sets of the aforesaid sferics or sferics files;
a suitable, broadband time-frequency level analysis 6 of the sferics data/data sets;
which is executed by means of software automatically 7 and/or
empirically 8; and
suitable measures of the sferic signal post-processing 9 (preferably graphically/optically incorporated—so as to be visible to the user—as an optimization routine, including level compensation, removing signal ranges and filtering out signal parts/technics etc.);
in order to generate according to the invention the preferred “fair-weather ideal signal” 10 (in this case, the intended singular or plural** alternating fields) which has minimal biotropic effect on the human body, among other things;
to which** in turn broadband is added (mixed into) 11, for example one or more aperiodic or, respectively, stochastic and/or periodic and/or
additional impulse-heavy signals (see noise, periodic impulses) 12 imprinted in a suitable level ratio (compared to sferics signal 10), and in the end the (data) signal end product is stored in the form of a file;
hereafter follows a digital-to-analog converter together with level/intensity control 13 (in back-coupling with 17);
in addition, a broadband, suitable signal power amplifier 14
that feeds a sferics/signal-transmitting antenna 15 and/or with this via cable feed lines or the like metallic connectors 15
an “ideal fair-weather field sferics” preferred according to the invention are generated directly to a human/an animal/a plant 16 directly via surface alternating field emission or radiation,
which in turn, in backcoupling with an EMF/alternating field detector 17 together with

receiving antenna or, respectively, measuring antennae element 18, scans or measures the ambient alternating field
in order to transmit a control signal to the level-controllable D/A converter 14, by which the “ideal fair-weather field/sferics” suitable according to the invention are suitably more intense than possibly present surrounding technics and, moreover, the existing emission time X [of] online weather fields/sferics in the form of their online sferics (which penetrate walls) having biotropic effects on living organisms.

In conclusion, these surrounding online sferics and technics according to the invention are thus masked or concealed by the synthetically emitted, level-amplified “ideal fair-weather field/sferics”, and thus are demonstrably (among other things) neuronally or physically ineffective or are concealed in the aforementioned tests.

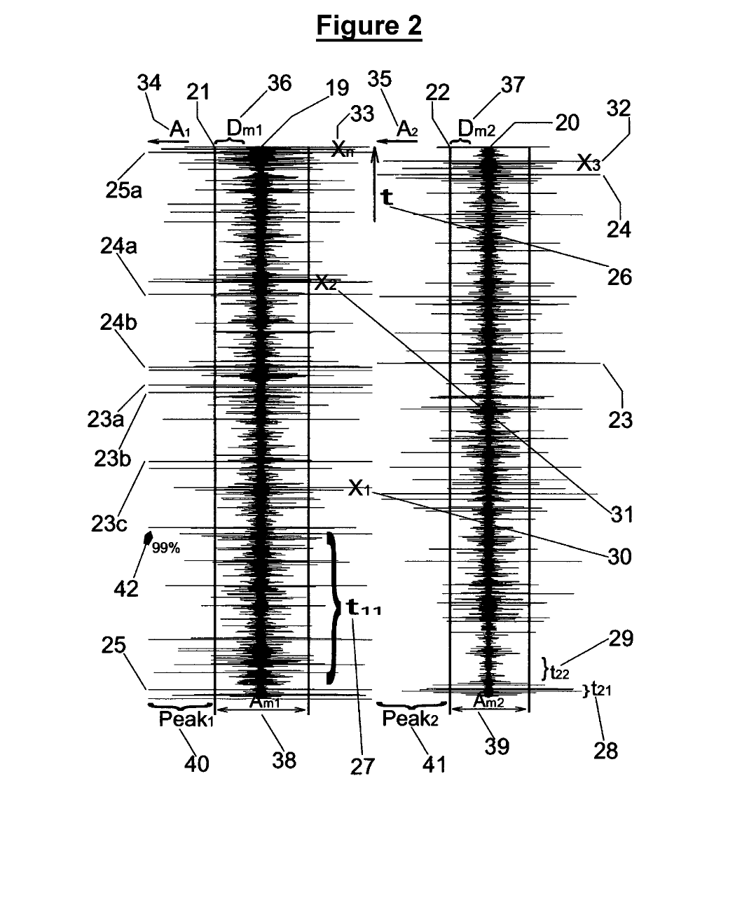

As is clear from the exemplary time signal/graphic representation according to FIG. 2, the following is intended to be summarized by a listing of reference numbers, including a brief description, their names/relevance:
19 and 20 represent an impulse-heavy alternating signal or sferics time signal path;
21, 22 represent an average, preferably sferic sequence/pulse level range;
23, 23a, 23b, 23c, 24, 24a, 24b, 25, 25a represent exemplary, too strong or too intense sferics or, respectively, individual atmospheric pulse discharges that, according to the invention, are removed or, respectively, eliminated/erased in the signal time curve;
26 represents the two exemplary, graphic sferis/signal curves visible or depicted over time t (see also time axis t);
27 t11 represents a partial sferic/signal curve range which manages without elimination/deletion of single pulses (according to X1 to Xn);
28 t21 represents a partial sferic/signal curve or range having several weak-level pulse discharges;
29 e t22 represents a partial sferic/signal curve or range having a multi-discharge single pulse of higher intensity, but still acceptable level range;
30 represents an exemplary, almost overmodulated, or high level but still acceptable, not too strong sferic/single pulse X1;
31 represents an exemplary, almost overmodulated, or high level but still acceptable, not too strong sferic/single pulse X2;
32 represents an exemplary, almost overmodulated, or high level but still acceptable, not too strong sferic/single pulse X3;
33 represents an exemplary, almost overmodulated, or high level but still acceptable, not too strong sferic/single pulse Xn;
34 represents the amplitude range (or, respectively, amplitude axis or axis label) that applies to the 1st graphic example of a sferic/pulse sequences depiction;
35 represents the amplitude range (or axis or axes) (or, respectively, amplitude axis or axis label) that applies to the 2nd graphic example of a sferic/pulse sequences depiction;
36 represents an average pulse sequence/sferics signal dynamic extent/range Dm1 (calculated assuming an amplitude range from 0 to 100%, in this case approx. 40%) of the exemplary sferic/pulse sequences drawing 1;
37 represents an average pulse sequence/sferics signal dynamic extent/range Dm2 (calculated assuming an amplitude range from 0 to 100%, in this case approx. 25%) of the exemplary sferic/pulse sequences drawing 2;
38 represents an average pulse sequence/sferics signal amplitude extent/range Am1 (up to here approx. 40% statistically aggregated/existing with regard to the amplitude value) of the exemplary sferic/pulse sequences drawing 1;
39 represents an average pulse sequences/sferics signal amplitude scope/range Am2 (up to here approx. 25% statistically aggregated/existing with regard to the amplitude value) of the exemplary sferic/pulse sequences drawing 2,
40 represents a stronger pulse/sferic signal value (starting here in terms of statistic amplitudes, fewer aggregated single pulses occurring having an amplitude value>40%, and as a whole more single pulses are recorded in a greater sferic dynamic range, consequently more suitable file) and the like;
41 represents a stronger pulse/sferics signal value (which if both +/−flanks of a single pulse are contacted as defined is still NOT considered overmodulated; (starting here in terms of statistic amplitudes, fewer aggregated single pulses occurring having an amplitude value>25%; as a whole, less suitable because the overall dynamic range of the overall fewer available individual sferic pulses is higher); and
42 represents the defined, absolute maximum pulse/sferics value which, if both +/−flanks of a single pulse are contacted as defined is considered overmodulated, so according to the invention it must be erased (in the general context of the invention, in terms of the alternating signal path/sferic pulse sequences, all single pulses are to be eliminated); however, at this reference character 42 is an example of a single pulse that only reveals a left-side amplitude edge at the representation maximum >99% (right side approx. 85%), which according to the context of the invention should not be erased or eliminated (cf. also Examples X1 30 to Xn 33).

According to FIG. 2, the further discussed time/amplitude-related signal representation/graphic area 19 (with according to FIG. 1 and the elements or, respectively, reference numbers 1 through 5) includes a recorded, less fluctuating, more suitable sferic signal on the part of the signal dynamic Dm1 (often in the “alternating time signal original state/direct recording state,” including “snuck-in” periodic technics modulators, which are only detectable in a spectrum analysis and must likewise be filtered out according to the invention) in a signal amplitude mode representation per axis marker A1 (see reference No. 34) over time axis t, further with subgraph 20 (similarly according to 19: often in the “alternating time signal original state/direct recording state,” including “snuck-in” periodic technics modulators, which are only detectable in a spectrum analysis and are to be filtered out according to the invention), in the signal amplitude mode per axis marker A2 (see reference No. 35) of the depicted, insufficient, biotropic sferics signal having very strongly fluctuating single pulses or, respectively, sferics signal dynamics Dm2 (adverse biotropic effect) from various points of origin alternating from near and far, wherein in this case in the signal curve 20:
Alternating signal amplitude ranges t21 and t22 already mark a removal or “disqualification” upon initial review of the quality of the sferics signal and the range of the sferics dynamics (see too strongly fluctuating impulse sequences according to time range t22 having less intensity compared to t21 along with too strong intensity in direct alternation);
Furthermore, the single-pulse examples X1, X2, and X3 through Xn (see reference Nos. 30, 31, 32, 33) [are] “quasi or almost overmodulated” or high-level but still are acceptably not too high/strong within the Peak 1/Peak 2 level range (see reference Nos. 40, 41), thus occupying a suitable sferic dynamic range (cf. level-exceeding ranges>maximum values according to “Peak 1” 40 and “Peak 2” 41).
Finally, the desired “average” sferics signal level (see dynamic/range Dm1 and Dm2 according to 36, 37) is characterized by +/−Dm2 having an intensity/level auxiliary line 22 of markedly lower intensity than those in the amplitude signal via auxiliary line 21 of subgraphic 19 with Dm1; accordingly, Dm1>Dm2.
Thus, on the other hand, a comparison of maximum values and average amplitude range of subgraphics bis 21 and bis 22 reveals the following: Peak 1<Peak 2 (see reference Nos. 40, 41) and Am1>Am2 (see 38, 39). In this representation of the range of amplitudes, single pulses are statistically less frequent (see lightning discharge points of origin according to geographically nearer, more intense single pulses 23, 23a, 23b, 23c, 24, 24a, 24b, 25, and 25a), which sometimes also are to be “disqualified/eliminated/deleted” (if they exceed the display range/measurement value/amplitude by more than 99% or as defined according to the invention, however; see overmodulation on account of the maximum level).
Although in principle 40 from 19 more maxima impulses are to be seen quantitatively in the amplitude intensity range “Peak 1,” as can be seen OUTSIDE the signal time range t11 (see reference No. 27), they are not so extreme in terms of overall impulse behavior as fluctuating, extreme value impulses. In principle, this time range t11 in signal sequence curve 19 “could” be eliminated since it fluctuates “at the borderline.” Both signal curves were “normalized” for purposes of comparison, i.e. the respective maximum impulse value (a specific peak/impulse) given in time/amplitude sub-curves 19 and 20 in each instance limits the maximum level in the overall signal modulation.
In summary, the sferics recording according to subgraph 19 is usable (has/had to be partially time-optimized per the aforementioned single-pulse excisions), but the signal curve according to 20 was unsuitable even though it did not contain any technics (see permanent, periodic sustained oscillations/signal components similar to one or more sinusoidal signals).
In an assessment discussion as to which of the “too strong pulses” should be deleted from the signal curve context according to the invention on a time basis, it should be noted that significantly more visible pulses (see number of impulses) can be recognized in the statistically fluctuating/variant sferics signal/impulse sequence curve in subgraphic 19 (left side) compared to 20 (right side). This is also clear from the labeling threshold called “mean pulse dynamics” or Dm1 36 (in this instance approx. 40% compared to the amplitude maximum value of 100%), which in terms of amplitude or level value is significantly higher than Dm2 37 (in this instance approx. 25% compared to the amplitude maximum value of 100%). Consequently, the single pulse at the time marker X3 32 is probably and only in this case too strong “individually to impulse sequence” for the sferics signal sequence depiction 20, and must be deleted!

The process or method according to the invention should be rewritten serially with individual processing steps as follows:
Broadband, enhanced recording 2 of sferics by means of an antenna device 1 at a suitable location with a minimal alternating technics field (detection and evaluation in accordance with 17, 18), including analog-to-digital conversion 4 and data storage 5 of the aforesaid sferics signals.
A signal analysis 6 of the sferics signal data recording of time/frequency level ratios (based on specific empirical values of the inventor regarding 8 and 7, automated via software) follows hereafter, as well as the removal 9 (highly significant in the method) of sferics signal moments that are unsuitable in time and frequency patterns. The inventor proceeded here in part from the patent application DE10354023A1 (see reduction of data gaps in the spectrum of sferics by the aforementioned technics [as] permanent-periodic signal components in a specific frequency range; must be filtered, but generate a frequency range level leak in terms of the individual sferics spectra or impulses), wherein in this instance the sferics original should have been reestablished syndetically without interference; this is a fundamental error because newly made sferics should not correspond to the “alleged natural ideal” but rather
are trimmed/relieved of specific single pulse[s], partially on the part of the sferics signal dynamics (based on elements 6, 7 and 8 as a negative feedback optimization process/procedure); this should NEVER happen by means of sound engineering dynamic compression programs because, as a result, the diffracted sferic single pulses are in each instance totally “equalized by one another” in relation to the point of origin/intensity and weather conditions passed through (see oscillation—period duration has NO REAL relation to the actual intensity/distance ratio of individual sferics and their points of origin far from the recipient); thus, unpredictable and “unknown” biotropic effects of spherics/weather on the body (see created by evolution over millions of years=reference signal, similar to Schumann resonance) can be produced by the creation of false sferic dynamics (in other words, this creates in turn technics to be avoided). In other words: this corresponds to an approximation of the technics to be avoided, i.e. “careless synthetization of alternating electromagnetic field generation via electrical loads and field transmitters etc.”
Finally, this “idealized sferics signal sequence” 10 generated according to the invention (see step 9) becomes a synthetic noise signal that is preferably uncorrelated/aperiodic, and thus injected without effect in a neuronally neutral manner into nerve pathways and/or a signal that is harmless to human genes (see Schuman[n] resonance with harmonics or other therapy signals), which is impressed or mixed into or modulated into, as well as finally stored as an end product or file (see steps 10+11+12) with which—after digital-to-analog conversion 13—a composite signal that can be suitably controlled (likewise via element 13) for amplification operating system created via an antenna 15 or surface radiator 16 at an organism, which corresponds to the at-rest, broadband, impulse-heavy, ideal alternating field signal according to the invention that is optimized to have minimal biotropic effect.
This signal 15 is from now on to be emitted by surface radiator 16 or, respectively, to be provided to humans, animals and plants as an alternating field (e.g. by means of a material/jacket/surface material or the like).
According to the invention, it is significant that it is preferably be noted that the alternating field emission of the “ideal fair-weather field noise mixture” 15 has an alternating field detector (see 17, 18), i.e. a low-frequency measurement/signal detector with a “feedback” analyzer function (see element procedure/subsequent paths in the loop according to elements 13>14>15, 16>18>17 and then again 13>14 etc.) that reverts back to and affects the overall alternating field level, whereby the level of the “ideal fair-weather field noise mixture” (see 15, 16) is adjusted higher in relation to “ambient sferics/technics”! Only in this way does an important basic physical/biomedical principle exist, based on the masking or concealment of unwanted, nowadays typical (see industrialization), at times markedly disruptive, alternating field occurrence; see masking of “technics transmitter based on electrical network loads, wireless signal transmission devices” or “ambient weather, including associated online weather radiation/sferics” by the aforesaid “ideal fair-weather field noise mixture” according to the invention.
It should also be emphasized that, although the procedure according to the invention or the present process can take place continuously from elements 1 through 16 (17, 18) as a complete process in order to create an alternating field environment with minimal biotropic effects for humans/animals/plants,
typically, steps 1 to 10/11/12 take place separately in a procedure (or, respectively, work steps, including subsequent storage of data in a file); afterward, based on the generation and reproduction of “ideal fair-weather noise mixtures” of any length of time, a looping of processes 13/14/15/16/17/18 takes place as specified by the file from 10 or, respectively, is preferably carried out in the data/signal processing.

Preferred application fields of the device according to the invention for the generation or emission of a natural alternating electromagnetic field close to the body exist in all urban-mechanized, daytime/nighttime spheres of life of humans/animals/plants and are listed below and briefly described:

Electrical devices such as, for example, all battery or mains-operated electrical devices or, respectively, trains/subways, motor vehicles or airplanes, clocks, listening devices and their accessories (see earphones and headsets), or modulating wireless transmitters or communications devices according to, among others, cellphones/WLAN/DECT telephones with additional signal emission via aforesaid ideal sferics, or directly on crops or animals as healing/growth stimulation, hospital beds/operating tables for the neutralization of artificial environmental technology radiation, computers/laptops/notebooks (technical alternating field emitter), 50/60 Hz mains/in-house/overhead power lines, and all additional electrical loads (e.g. stoves, washing machines, illumination/lamps etc.); finally, this also happens by means of clothing or clothing elements! Thus, the technical realization of the invention relates to a method and additional devices for the generation of alternating sferic field emissions with minimal meteorotropic or biotropic effects on humans, in the following abbreviated as BMSE.

According to the invention, in addition to the dependent claims it turned out
that an impulse-heavy, broadband alternating field of natural atmospheric origin, tapped by receiving devices, with minimal technical interfering signal components of human origin—called sferics 1—is trimmed of unusable, too-strong (according to the impulse amplitude intensity) individual extreme value impulses/sferics 6, 7, 8, and 9 or, respectively, is relieved of these by removal via signal engineering over its temporal existence, and moreover is 11 preferably mixed together with at least one synthetic aperiodic and/or synthetic periodic and/or artificial stochastic broadband signal 12, and finally is emitted to organisms via electrically conductive transmission antenna devices in a manner that is controllably intensity-amplified (with regard to the overall alternating signal level) 13, 14 and 15 compared to the surrounding natural, weather-related ambient sferics and technical, man-made industrial/electronic interference fields 17, 18;
that a recorded, selected, alternating atmospheric signal (called fair-weather sferics 1) of natural origin is recorded and stored during fair-weather conditions, which corresponds to the requirements of meteorological high pressure and is crucially dominant in the recording environment;
that, in a defined time signal section having an average sferic/impulse signal dynamic range, the fair-weather sferics signal is relieved of one or more individual sferics pulses whose levels are too strong or too high in terms of statistical intensity, and thus an extraction or removal 6, 7, 8, 9 of these individual impulses takes place in the fair-weather sferics signal; in connection with this, it is to be emphasized that one or more impulse signal sequences/sferics files of natural origin are first “reasonably” level-adjusted across the entire timespan of the file (for the most part, it is necessary to amplify the file by, for example, +1.6 dB or another decibel in order to achieve what is known as a level-adjusted “file normalization state” (identically amplified or the like over the entire timespan, from the starting time A until after B); see level adjustment state; “normalization” is the known term according to sound studio standards), and only thereafter is the removal of fragmentary, level overriding/adverse sferic/single pulses (cf.

moments in time reference characters and their unusable single pulses (23, 23a, 23b, 23c, 24, 24a, 24b, 25, 25a), logical or, respectively, contemplated according to the invention;
that the fair-weather sferics signal 1 arises from a meteorologically defined weather condition like the one created by wide-ranging areas of ambient high pressure, and for that reason a striking impulse occurrence probability dominance by uniformly dynamic far-field sferics of median intensity (>5,000 KM distance), as well as median-dynamic, stronger-intensity, average far-field sferics (>1,000 KM distance), and in particular fewer or preferably no unusable near-field sferics or dark-field discharges from storms or, respectively, low-pressure areas in the near field (<500 KM) are accommodated or included;
that a noise-like signal 12 at a suitably level ratio 11 is admixed to the fair-weather ideal sferics signal 10;
that the self-regulation of the ideal fair-weather sferic noise alternating field can be deactivated and thus can be regulated by hand; and finally
that one or more different ideal fair-weather sferics/noise alternating field files are randomly appended to one another before they are fed to the digital-to-analog converter, together with the level-variable alternating field amplification process 13, 14, 15, 16, 17, 18.

Concluding remarks of the inventor: the suitable selection of weather conditions, if a suitable location for a recording of “fair-weather sferics” that is for the most part undisturbed technics actually exists, is in itself already a challenge to find nowadays in our mechanized or, respectively, civilized environment. Moreover, the selection of a “reference sferics recording” as a “post-processing file” according to the invention, preferably from a plurality of meteorologically as well as geographically favorable sferics recordings, is important in order to find “the right one.” A certain knowledge or routine after more than 15 years of sustained searching, or searching definable in terms of the physical sciences, for such a “recording” is also guaranteed with a sense of “experienced hearing” as to how a fair-weather sferics impulse sequence sounds “good,” as if one just started like that or copied it from the inventor; know-how! Likewise, the process steps according to the invention are joined or linked, which in the context of the invention must be taken into account during optimization of the given file for too-strong (exceeding 99% peak) impulses, which eliminates “hearing,” in order to create an IDEAL. Finally, the suitable file/signal amplification or leveling counts among this (moreover/reiterated, possible removal of other sferic impulses), which appropriately leads to an additional IDEAL fair-weather file and/or suitably to at least one (a)periodic, broadband signal component to be mixed as a file (into an achieved overall file), whereby a maximum “bio/human effect neutrality” of the possible alternating signal file is offered for an alternating field emission to organisms according to the invention—a challenge in itself, in addition to the not-meaningless radiation form of sferics (which in the end must be recognized by meteorology and physics) applied to “patients” and “people in need” (see not hypochondriacs but sufferers of medically detectable and causally correlatable ailments, like the one applied in the aforementioned weather-sensitivity study, among others, POSITIVELY by means of a portable sferics/alternating field/small radiation device[)] bore “fruit”! The 1:1 or direct use of sferics recordings only repeats sferics already recorded along with technical ambient forms of radiation (see technics/signal components to be filtered out as periodic “permanent sferics destroyers” for each occurring frequency range), perhaps during good weather conditions, which are “perforated” by technics, etc.; biomedically questionable facts of the application!

With regard to the “gift of nature”: certain sought-out alternating signal/impulse signal sequences 19 of high quality with regard to statistical variants, individually thus extremely fluctuating sferic time/level/frequency modulation patterns and their respective stochastic occurrence probabilities is nearly perfect for establishing an optimally neutral, biomedically [and] biophysically ineffective, de facto relaxing alternating signal or alternating field; a good basis for the present invention and a gift of nature, if one searches long enough! The aforementioned sub-aspect of “sferics modulation” means the statistically highly variant ionospheric reflection design shaping of often-occurring single-pulse, low-frequency envelopes/modulations having different (modulation) eigenfrequencies starting at a few multiples of 10 Hz into the range of thousands of kilohertz (like a comb filter structure incorporated into a single pulse!).

1. A method for generating sferics alternating field emissions having minimal biotropic effect (BMSE) for optimized application of alternating field-type radiation to humans, animals or plants, characterized in that one or more alternating signals attributable to or based on natural, atmospheric impulse discharge processes (1) are post-processed (6, 7, 8, 9) in their signal curve forms (1, 2, 3, 4, 5, 19, 20, 34, 35),
wherein interfering, periodic duration/signal components of technical origin are deleted as well as individual impulses that are too intensive in signal dynamic and level-related terms (23, 23a, 23b, 23c, 24, 24a, 24b, 25, 25a);
wherein said post-processed alternating signals (10) are mixed or admixed (11), preferably with multiple selected, aperiodic/periodic/impulse-heavy alternating signal forms (12) in favor of an overall alternating signal (10, 11, 12); and
wherein the post-processed alternating signal (10, 11, 12) is radiated or, respectively, emitted (15) via alternating field transmission means (13, 14, 17) to humans, animals or plants in a level-controllable manner.

2. The method of claim 1, wherein the signal processing is on digital domain in time/frequency level range under generation of sferics signal sequence files whose amplitude modulation range is suitably leveled to average intensity across the entire file timeframe—similar to a file-level normalization—and afterwards are subject to a time-frequency level analysis (6), from which information is gained on undesired near-field sferics having a too strong a level of sferics/single pulses (23, 23a, 23b, 23c, 24, 24a, 24b, 25, 25a) which are removed or, respectively, extracted from the sferics signal sequence files at their respective signal timeframe positions.

3. The method of claim 1, wherein broadband sferics signals which are essentially physically, psychically, biotropically or electrosensitively neutral or which have no effect on humans, animals or plants are created, at least one dynamic, time-processed fair-weather ideal sferics signal (9) or fair-weather ideal sferics signal section (27) having at least one broadband, stochastic, thus noise-like signal (11) and/or aperiodic and/or periodic and/or periodic impulse-like signal is provided for application onto humans, animals or plants in a suitable level ratio (13).

4. The method of claim 1, wherein suitable, preferably automatic leveling of additive technologically/synthetically produced alternating fields (10) without medical-meteorological effects (ideal fair-weather sferics alternating fields) is provided that cover natural ambient weather-related sferics and artificial man-made or urban alternating field signal mixtures or, respectively, technics, wherein an ambient alternating field signal receiver (18) scans the ambient overall signal mixture based on field intensity (17), and an ambient alternating field signal transmitter (14, 15) generates the synthetically produced and intensity-controllable alternating field signal mixture (15) from fair-weather ideal sferics with a stochastic/noise-like signal (16) and/or aperiodic and/or impulse-like signal part additively and with adapted level (14, 15).

5. The method of one of claim 1, wherein an ambient radiation scanning device for suitable, preferably automatic leveling of additive, technologically/synthetically produced alternating fields (10) without medical-meteorological effects (ideal fair-weather sferics alternating fields) that cover natural ambient weather-related sferics and artificial man-made alternating field signal mixtures or, respectively, technics, is provided, wherein a comparator or comparison measurement average over a certain timeframe is calculated from the ambient alternating field signal receiver (17, 18).

6. The method of claim 5, wherein the comparator or comparison measurement average (17) over a certain timeframe serves as the default emission level value for the intensity-based, time-averaged leveling (13, 14) of the (ideal fair-weather sferics noise) alternating field transmitter (15).

7. The method of claim 6, wherein the average leveling (13, 14) is done following the instruction that the intensity (15, 16) of the ideal fair-weather sferics noise alternating field (10) is preferably greater than that of the natural ambient weather-related sferics (18) as well as the artificial, man-made, i.e. industrial/electrical or, respectively, telecommunications alternating field signal mixture based on wireless communications technology and its low-frequency modulators, i.e. what are known as technics.

8. The method of one of claim 5, wherein the comparatively higher intensity setting (13, 14, 15) of the ideal fair-weather sferics noise alternating field (10) is preferably automated, i.e. it regulates itself over a certain period of time and/or is activated or, respectively, controlled or, respectively, regulated by an external control signal of a mobile communications device.

9. The method of claim 8, wherein one or more ideal fair-weather sferic/noise/alternating field files are linked to one another in a loop before they are fed to athe digital-to-analog converter together with [the] level-variable alternating field enhancement process (13, 14, 15, 16, 17, 18).

10. The method of claim 1, wherein detecting, recording, post-processing and storing sferics signals (1, 2, 3, 4, 5, 6, 7, 8, 9) is independent or separate from and time-independent of the process or, respectively, method of utilization, and so that the alternating field emission takes place in the form of level-controlled, ideal fair-weather sferics/noise generation (10, 11, 12, 13, 14, 15, 16, 17, 18), which are stored and can be retrieved at any time.
